(19) United States
(12) Patent Application Publication
(10) Pub. No.: US 2025/0057682 A1
(43) Pub. Date: Feb. 20, 2025

(54) UNIVERSAL WRAPAROUND KNEE BRACE

(71) Applicant: Nicholas Robert DeMaria, Fort Myers, FL (US)

(72) Inventor: Nicholas Robert DeMaria, Fort Myers, FL (US)

(21) Appl. No.: 18/807,075

(22) Filed: Aug. 16, 2024

Related U.S. Application Data
(60) Provisional application No. 63/533,471, filed on Aug. 18, 2023.

(51) Int. Cl. A61F 5/01

(52) U.S. Cl. CPC A61F 5/0125

(57) ABSTRACT

A universal wraparound knee brace includes a central body section having a concavely curved inside surface for engaging and extending vertically across the wearer's knee. A corresponding pair of first and second upper flaps extend from respective opposing vertical edges of the central body section and a pair of corresponding first and second lower flaps similarly extend from the opposite vertical edges of the central body section. The second upper and lower flaps carry respective interior D-rings. Each first flap is engaged with a respective interior D-ring attached to its corresponding second flap and is pulled through the D-ring in a reverse direction so that corresponding first and second flaps wrap around the wearer's leg above and below the knee. The corresponding first and second flaps are secured in place by complementary first and second hook and loop connectors. Each second flap also carries an elongate locking strap and a respective exterior D-ring attached at an inner end of the locking strap. Each locking strap is wrapped around a respective pair of interengaged first and second flaps, inserted through a corresponding exterior D-ring, pulled in a reverse direction until snug and secured to itself by third and fourth hook and loop connectors. This provides the brace with upper and lower means of bi-directional tightening so that the brace is securely custom fitted to the wearer and resists unwanted migration relative to the knee.

### RELATED APPLICATION

This application claims the benefit of U.S. Provisional Application Ser. No. 63/533,471 filed Aug. 18, 2023.

### FIELD OF THE INVENTION

This invention relates to a universal wraparound knee brace and, more particularly, to an offloading knee brace that can be used conveniently and effectively by persons suffering from osteoarthritis and patients recovering from knee surgery or injuries to the knee.

### BACKGROUND OF THE INVENTION

Orthopedic knee braces are widely utilized by persons suffering from chronic knee pain and patients recovering from knee surgery or injuries to the knee and surrounding tissues and ligaments. Wraparound knee braces are often prescribed for persons experiencing painful osteoarthritis of the knee. Such orthotics are specifically constructed to offload stress and pressure from arthritic regions of the knee and patella and thereby reduce the wearer's pain.

Traditional wraparound knee braces have employed four adjustable straps that wrap around the wearer's leg, typically with two straps positioned above and two positioned below the knee joint. Such braces often provide an uncomfortable and unsatisfactory fit. When the wearer is active, the brace is apt to migrate relative to the knee, which can interfere with the intended beneficial offloading. Gels have been applied to the interior surface of these braces to reduce unwanted movement and displacement of the brace relative to the knee. However, such gels have not proven to work very effectively. Moreover, it can be inconvenient, tedious and time consuming for the wearer to properly engage and tighten the four wraparound straps. A further shortcoming is that many of these existing braces are designed to provide the wearer with a custom fit. Accordingly, a wide variety of sizes must be manufactured to fit all or at least most sizes of wearers. Prospective wearers must evaluate, be fitted for and select a correctly sized brace.

Previous attempts to introduce a universal wraparound knee brace for osteoarthritic patients have not been optimally successful. One such existing universal knee brace employs flexible upper and lower panels that wrap around the wearer's knee generally above and below the patella, respectively. The panels are secured in place by complementary hook and loop (e.g., Velcro™) connectors. A top strap extends around each panel and is engaged with a D-ring and locked in place, again with complementary hook and loop fasteners. This brace still fails to provide an optimally secure fit that reduces undesirable shifting and migration of the brace along the user's leg and knee, particularly during vigorous activities and exercise. The existing universal wraparound knee brace also features an external condyle pad mounted exteriorly on the brace for indirectly engaging and applying pain-reducing pressure to the wearer's medial or lateral femoral condyle adjacent the knee. This condyle pad does not directly engage the knee and is not adjustable in thickness. As a result, it is not likely to optimally fit or provide therapeutic pain relief to the particular person wearing the knee brace.

### SUMMARY OF THE INVENTION

It is therefore an object of the present invention to provide a universal wraparound knee brace that is readily adaptable for use by persons of various sizes.

It is a further object of this invention to provide an improved wraparound knee brace that is more securely and reliably attachable to the wearer's leg and knee and which effectively resists undesirable movement and migration along the leg and relative to the knee.

It is a further object of this invention to provide a wraparound OA knee brace that is especially effective for use by persons suffering from painful osteoarthritis.

It is a further object of this invention to provide a wraparound knee brace featuring an internal frame which is ergonomic, comfortable to wear and aesthetically appealing.

It is a further object of this invention to provide a universal wraparound knee brace that is constructed to effectively unload undue pressure on the patella and crease behind the knee to reduce the wear's knee and leg pain.

It is a further object of this invention to provide a wraparound knee brace that is quick, convenient and easy to attach to and remove from the knee.

It is a further object of this invention to provide a wraparound knee brace that provides for a secure and comfortable custom fit.

It is a further object of this invention to provide a universal wraparound knee brace featuring an internal frame enclosed within a preferably one-piece soft padded cover, which is more ergonomic, aesthetically attractive and comfortable to wear than existing knee braces of this type.

It is a further object of this invention to provide a wraparound knee brace employing an improved condyle pad mounted interiorly on the brace for directly engaging and better relieving pressure on the wearer's femoral condyle, and which features adjustable positioning and thickness for effectively customizing the brace to optimally fit a particular wearer.

This invention features a universal wraparound knee brace that is particularly suited for use by persons suffering from osteoarthritis of the knee. The brace is also designed to effectively unload undue pressure from regions of the knee such as the patella and the crease in the back of the knee. The brace includes a flexible body having a central section for engaging and extending vertically across a side of the wearer's knee. Corresponding first and second upper flaps are attached to and extend outwardly from opposing first and second vertical edges respectively of the central body section. Corresponding first and second lower flaps are likewise attached to and extend outwardly from the first and second vertical edges respectively of the central body section below the upper flaps. A first concave recess is formed between the first upper and lower flaps and a second concave recess is formed between the second upper and lower flaps. Each first flap includes an outer surface that carries a first connector. The first connector is releasably interengageable with a complementary second connector carried by at least one of the outer surface of the first flap and an outer surface of the central body section. Each second flap has an outer surface that supports a respective interior D-ring. An upper locking strap is attached proximate an inner end thereof to the outer surface of the second upper flap. A lower locking strap is similarly attached proximate an inner end thereof to the outer surface of the second lower flap such that the upper and lower locking straps are extendable outwardly from the respective second flaps and generally parallel to one another. Each locking strap has a respective exterior D-ring attached to the inner end of the locking strap. Each locking strap has an outer surface that carries a respective, complementary pair of releasably interengageable third and fourth connectors.

To attach the knee brace to the wearer's knee, the central body section is engaged with the wearer's leg by engaging a concave inner surface of the central body section against the wearer's knee such that the first upper flap and corresponding second upper flap are disposed above the knee and the first lower flap and corresponding second lower flap are disposed below the knee. Each first flap and its corresponding second flap are wrapped about the wearer's leg and releasably interengaged. Specifically, each first flap is inserted through the interior D-ring supported by the corresponding second flap and pulled snugly in a reverse direction to wrap around the wearer's leg. The complementary first and second connectors carried by the outer surface of the first flap and/or the central body section are pressed together and releasably interengaged. This operation is completed for both corresponding pairs of first and second flaps. As a result, the corresponding first and second upper flaps are secured together and wrapped about the wearer's leg above the knee. Likewise, the complementary first and second lower flaps are secured together and wrapped about the wearer's leg below the knee. The upper and lower locking straps are then wrapped exteriorly around the interengaged first and second upper flaps, and interengaged first and second lower flaps respectively. Each locking strap is inserted through the respective exterior D-ring attached to that strap and then pulled in a reverse direction to provide a snug fit with the wearer's leg both above and below the knee. The third and fourth connectors carried by each locking strap are then releasably interengaged to lock the brace closed and secure the upper and lower locking straps, as well as the underlying interengaged upper and lower flaps in place about the wearer's leg above and below the knee respectively.

In a preferred embodiment, the body of the knee brace may incorporate an internal frame composed of pliable material and covered by a padded or cushioned fabric exterior. A hinge may be mounted to the outside surface of the central body section for enabling the brace to be longitudinally bent in accordance with desired angular positioning of the knee. The outer surface of the central body section may include upper and lower pockets wherein upper and lower pivotally interconnected sections of the hinge may be mounted to respective internal frame elements of the brace. The complementary releasably interengageable connectors may include hook and loop (e.g., Velcro™) fastening components.

An interior surface of the central body section may carry a condyle pad mounting bracket that may be attached through the central body section to the exterior hinge. One or more condyle pads may be mounted to the mounting bracket interiorly of the central body section. A two-part, e.g., hook and loop, connector may be used for securing the pads to the mounting bracket. Pads of various thicknesses may be selected and employed to exert the desired pressure upon the femoral condyle. The pad may be enclosed in a flexible pouch, case or bag attached to the mounting bracket.

The first and second flaps may be wider than the locking straps. In such versions, the interior D-rings accommodating the first flaps may be wider than the exterior D-rings accommodating the locking straps. Respective interior and exterior D-rings may be mounted and arranged adjacent and generally parallel to one another.

### BRIEF DESCRIPTION OF THE DRAWINGS

Other objects, features and advantages will occur from the following description of a preferred embodiment and the accompanying drawings, in which:

FIG. 1 is an exterior plan view of the universal wraparound knee brace in an open condition;

FIG. 2 is an interior plan view of the knee brace in the open condition;

FIG. 3 is a fragmentary elevational view depicting the manner in which a representative one of the first flaps is interengaged with a corresponding interior D-ring carried by a corresponding second flap;

FIG. 4 is a fragmentary perspective view of the brace and particularly indicating the manner in which a representative locking strap is interengaged with a corresponding exterior D-ring attached to an inner end of the strap;

FIG. 5 is a rear perspective view of the knee brace in a closed condition wrapped around a wearer's knee and specifically illustrating the manner in which corresponding first and second flaps are interengaged and respective upper and lower locking straps are wrapped about the interengaged flaps and secured to hold the flaps and the knee brace in place about the wearer's leg and knee;

FIG. 6 is an alternative rear perspective view of the brace in the closed condition with the corresponding first and second flaps interengaged and the locking straps locked in place;

FIG. 7 is an elevational side view of the brace in the closed condition; and

FIGS. 8-10 are elevational side views of the knee brace depicting respective interior condyle pads having various thicknesses for providing custom therapeutic pain relief to individual users.

### DETAILED DESCRIPTION OF PREFERRED EMBODIMENTS

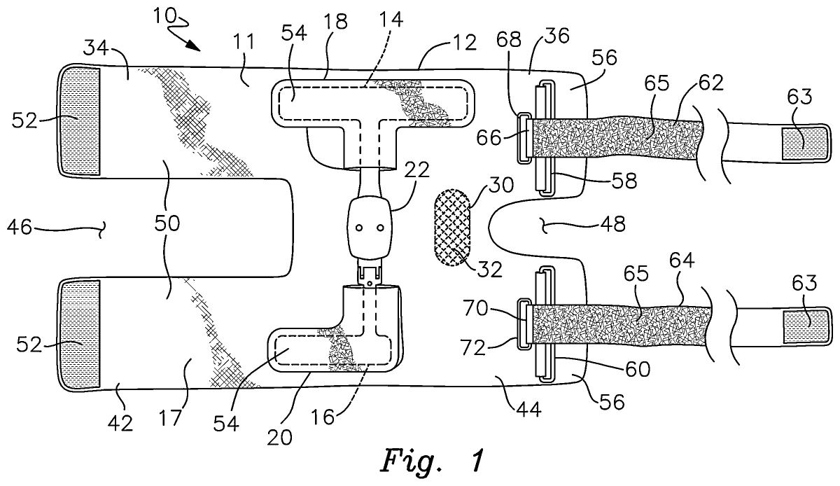

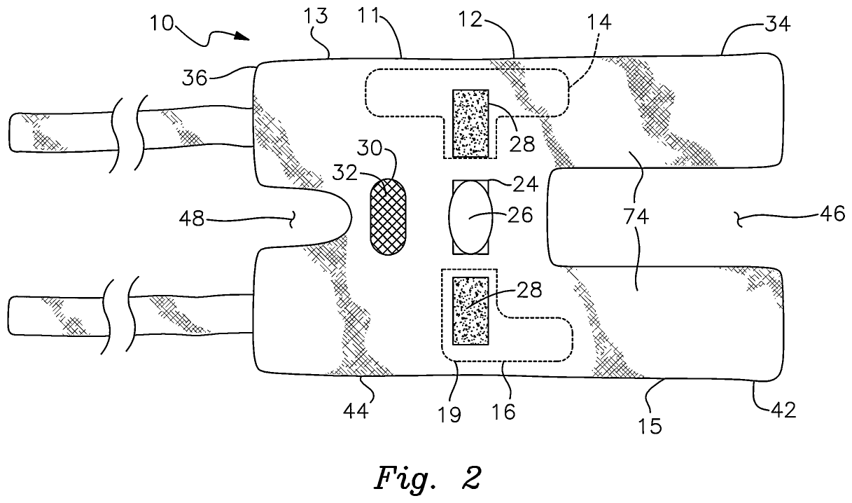

There is shown in FIGS. 1 and 2 a universal osteoarthritis (OA) wraparound knee brace 10 that is particularly effective for treating persons suffering from OA of the knee. The brace may also be used beneficially by patients recovering from surgery or knee injuries, as well as persons suffering from chronic knee pain and other medical conditions affecting the knee and surrounding ligaments and tendons. The brace may include one or more features for offloading or reducing painful pressure on the knee. The particular medical or therapeutic purposes for which the brace may be employed are not limitations of this invention.

Brace 10, which is illustrated in an open condition in FIGS. 1 and 2, is composed of a padded or cushioned material typically comprising a natural or synthetic flexible fabric body 11 that will be comfortable for persons wearing the brace. Body 11 preferably employs a universal single piece (unitary) design although in alternative embodiments, the body may comprise multiple distinct pieces. More particularly, brace body 11 includes a central body section 12 that extends longitudinally between an upper edge 13 and a lower edge 15 of the brace body. The exterior surface 17 of body 11 carries upper and lower pockets 18 and 20 having respective openings that are formed within the central body section 12 and, more particularly, are stitched or otherwise secured to the exterior surface 17 of body 11. Pockets 18 and 20 receive and hold the opposing pivoting arms of an adjustable hinge 22. This hinge may comprise various types of conventional hinges used in a known manner for maintaining the knee brace and the wearer's knee at a selected angle. An upper pivoting arm of hinge 22 is attached to an internal upper frame element 14 disposed in pocket 18 and in a lower pivoting arm is attached to an internal lower frame element 16 received by lower pocket 20. Each frame element comprises a relatively rigid, but somewhat pliable plastic or lightweight metal material that has a self-sustaining shape and a concave inner surface facing the interior side of the brace shown in FIG. 2 and extending laterally across the central section 12 of the body 11. These frame elements provide brace 10 with a fully internal supportive framework that is both ergonomic and aesthetically appealing. The pliable concave configuration of frame elements allows the brace to be securely and comfortably engaged with the wearer's leg and knee, as described below.

As shown in FIG. 2, the interior fabric surface 19 of central body section 12 carries a condyle support plate 24 that is secured by rivets, screws or other fasteners to the back of hinge 22 mounted on the opposite exterior surface of the brace body. Plate 24 in turn supports a therapeutic condyle pad 26 for engaging the condyle or other parts of the knee joint and leg to relieve painful pressure typically associated with OA and other knee ailments. Condyle pad 26 is constructed in a unique manner as described more fully below. The inner surface of central body section 12 also includes gel pads or strips 28 for reducing unwanted movement of the brace along the leg of the wearer. As shown in FIGS. 1 and 2, the brace may also include a patella receptacle 30 formed through brace body 11. Receptacle 30 may comprise a round or oval hole formed through the brace body and accommodating a thin elastic web or mesh 32. When the brace is worn by a patient or other person suffering from OA or a knee injury, the receptacle 30 and elastic web 32 support the wearer's knee such that pressure is offloaded from the front of the knee and pain is reduced.

A pair of corresponding first and second upper flaps 34 and 36 are integrally attached to and extend outwardly in opposite directions from opposing vertical edges of central body section 12. Likewise, a corresponding pair of first and second lower flaps 42 and 44 are integrally attached to and extend outwardly in respective opposite directions from opposing first and second vertical edges of central body section 12. The first flaps 34, 42 are longer and narrower than the second flaps 36, 44. As illustrated in FIGS. 1 and 2, this defines a first concave recess 46 between first flaps 34 and 42 and a second concave recess 48 between second upper and lower flaps 36, 40. The first flaps 34, 42 and second flaps 36, 40 are again typically composed of a durable, yet soft and comfortable padded or cushioned material, as is employed by the outer covering of the main body section. Natural or synthetic materials may be employed. As best shown in FIG. 1, pockets 18 and 20 that accommodate respective frame elements 14 and 16 extend partially across first flaps 34 and 42 respectively. Otherwise, the flaps lack the internal frame elements featured in the central body section 12 and therefore exhibit considerable flexibility that permits the flaps to be wrapped easily around the leg of the wearer and releasably interengaged in a manner described more fully below.

Each upper and lower first flap 34, 42 includes an outer surface 50 that carries a first connector 52 proximate a distal end of the flap. Complementary second connectors 54 are carried on the outer surfaces of upper and lower pockets 18 and 20. Each second connector 54 is releasably interengageable with a respective first connector 52. Preferably respective connectors 52 and 54 comprise respective first and second parts of a conventional two-part releasable fastening system such as a hook and loop (e.g., Velcro™) fastener.

Each second flap 36, 44 has an outer surface 56. An upper interior D-ring 58 is attached by stitching, adhesive or other secure fastening means to outer surface 56 of upper second flap 36. A lower interior D-ring 60 is similarly secured to outer surface 56 of second lower flap 44. An elongate upper locking strap 62 is stitched or otherwise fastened at a proximal end thereof to outer surface 56 of flap 36. A lower locking strap 64 is similarly attached proximate an inner end thereof to outer surface 56 of second lower flap 44. The upper and lower locking straps 62 and 64 are extendable outwardly from respective second flaps 36 and 44 in the generally parallel manner depicted in FIGS. 1 and 2. Upper strap 62 has a sleeve 66 formed at its inner end. An upper exterior D-ring 68 is engaged with sleeve 66 and thereby secured to the inner end of strap 62. By the same token, a sleeve 70 is formed by the inner end of lower locking strap 64. Sleeve 70 interengages a lower exterior D-ring 72 and thereby secures that D-ring to lower locking strap 64. The upper first and second flaps and their corresponding interior D-rings 58, 60 are wider than the locking straps 62, 64 and their exterior D-rings 68,72. Although the term “D-ring” is used herein, it should be understood that various other types and shapes of rings and grommets may be employed in an analogous manner within the scope of this invention. The term “D-ring” should be understood to encompass all such devices. Each of straps 62 and 64 includes a third connector component 63 on its outer surface proximate a distal end of the strap and a complementary fourth connector component 65 spaced longitudinally inwardly on the upper surface of the locking strap. The complementary third and fourth connectors 63 and 65 again preferably comprise respective complementary hook and loop components or alternative fastening means.

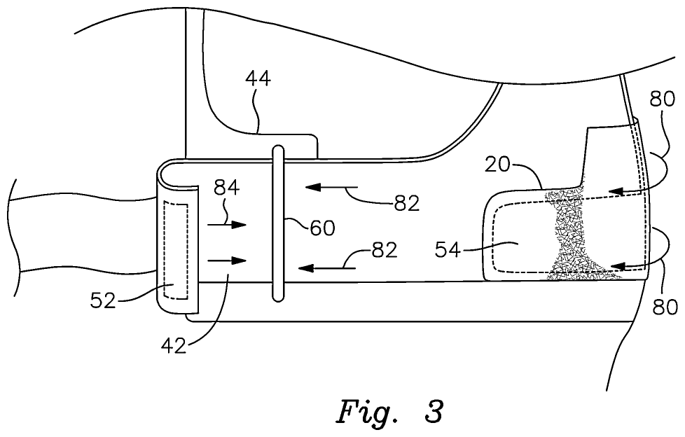

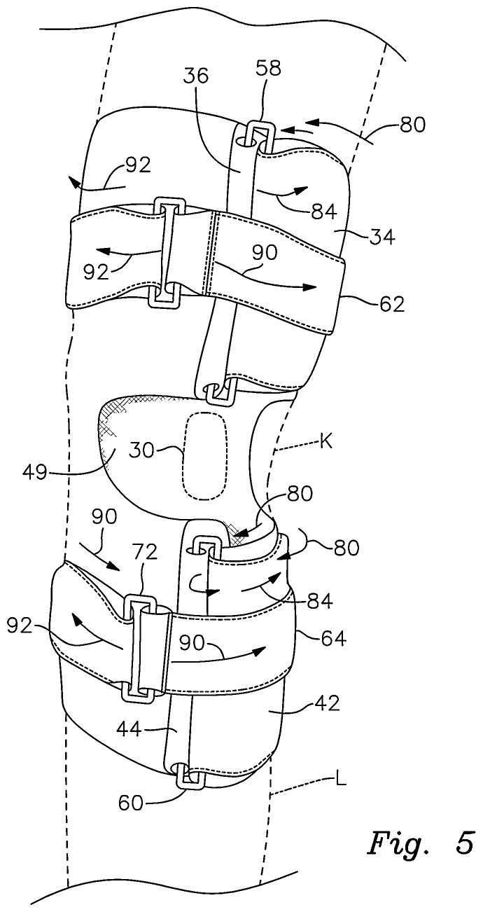

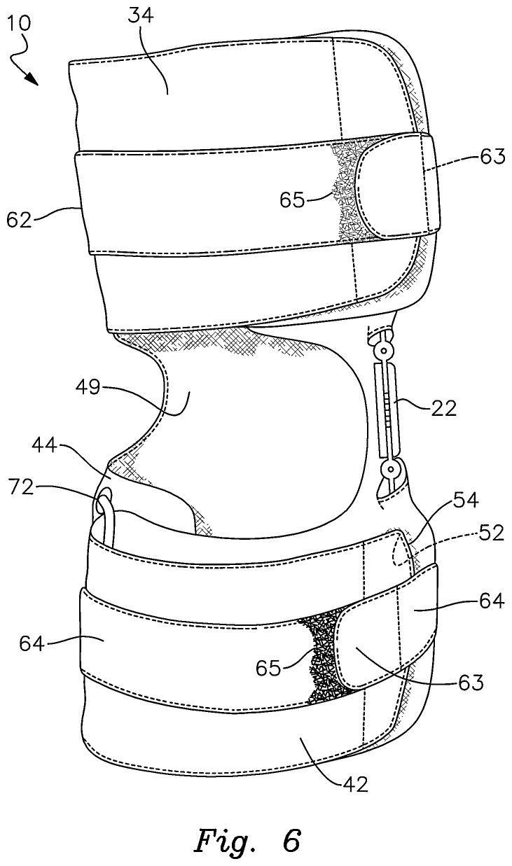

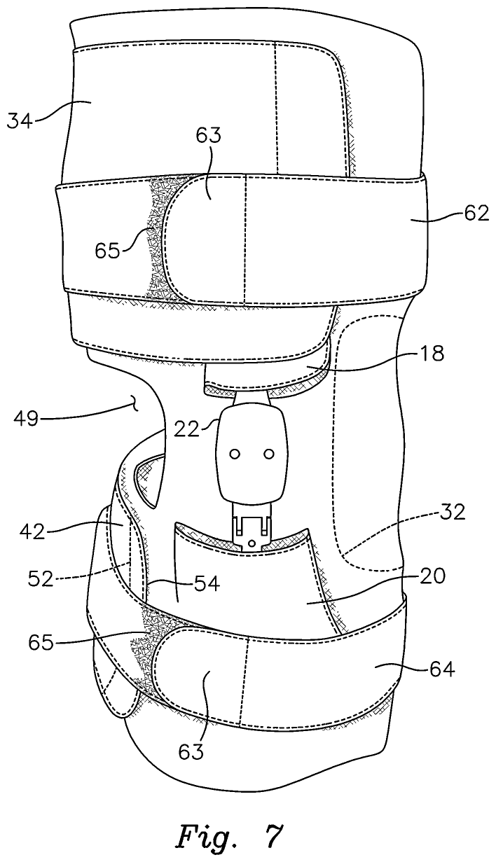

Knee brace 10 is attached to the wearer's knee and secured in place in the following manner. The central body section 12 is engaged longitudinally against the wearer's leg by placing the concave internal frame elements 14, 16 within pockets 18, 20 against the wearer's knee such that first upper flap 34 and corresponding second upper flap 36 are disposed above the knee and first lower flap 42 and corresponding lower second flap 44 are placed below the knee. Brace 10 should also be oriented such that exterior hinge 22 and interior condyle pad 26 are properly positioned relative to a selected side of the wearer's knee. The wearer's patella should be accommodated by webbed receptacle 30. The corresponding first and second flaps, as well as the upper and lower locking straps are then respectively interengaged as shown in FIGS. 3-7, to secure the brace in place around the wearer's leg and knee. Initially, corresponding first and second upper flaps 34 and 36 are wrapped about the wearer's leg above the knee and releasably interengaged; the corresponding first and second lower flaps 42, 44 are likewise wrapped about the wearer's leg below the knee and releasably interengaged. As represented by arrows 80 in FIGS. 3 and 5, representative first lower flap 42 is wrapped around and encircles the wearer's leg (see leg L and knee K shown in phantom in FIG. 5). The distal end of flap 42 is inserted in the direction of arrow 82, FIG. 3, through interior D-ring 60 carried by corresponding second lower flap 44. Flap 42 is pulled through and around D-ring 60 and is drawn in the reverse direction indicated by arrows 84 to interengage first flap 42 with corresponding second flap 44. Flap 42 is pulled rearwardly until the interengaged straps 42 and 44 snugly and securely wrap around the wearer's leg below the knee. As shown in FIGS. 3, 6 and 7, first hook and loop component 52 is pressed against the second hook and loop component 54, which fastens the first flap 42 and interengaged second flap 44 securely in place and fully encircling the leg below the knee. First upper flap 34 is interengaged with corresponding second upper flap 36 and upper D-ring 58 in a similar manner. Specifically flap 34 is likewise pulled rearwardly through D-ring 58 as shown by arrow 84, FIG. 5, to snugly encircle interengaged upper flaps 34 and 36 about the wearer's leg L above knee K. First upper flap 34 is secured in place again by interengaging its complementary hook and loop fasteners to securely encircle the wearer's leg above the knee. When the flaps are closed, concave recesses 46 and 48 in body 11 define an opening 49, FIGS. 5-7, which alleviates pressure and resulting pain at the back of the knee.

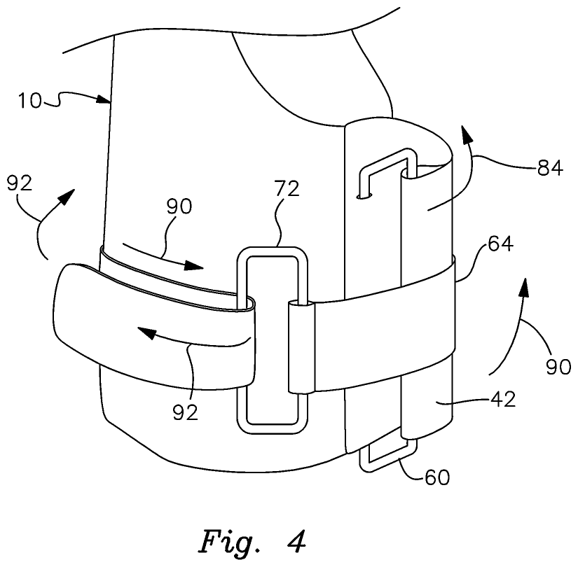

After the corresponding upper and lower flaps are interengaged in the above-described manner, the brace is releasably locked in place by engaging the upper and lower locking straps 62 and 64. As shown by representative lower locking strap 64 in FIGS. 4 and 5, the strap is wrapped over the interengaged first flap 42 and second flap 44 and encircles brace 10, as indicated by arrow 90. Strap 64 is then inserted through corresponding exterior D-ring 72 and pulled in the reverse direction indicated by arrow 92. When a desired tightness/snugness is achieved, the upper tightening strap is secured in place by pressing third and fourth connectors 63, 65 together or otherwise releasably interengaging connector 63 with fourth connector 65.

The upper locking strap 12 is likewise wrapped about its interengaged first and second flaps 34, 36 and is secured in place in a similar manner to lower strap 64. In each case, the locking strap 62, 64 is pulled snugly in a reverse direction through its corresponding exterior D-ring and the third hook and loop connector 63 proximate the distal end of the strap is pressed against the complementary fourth connector 65 carried by an inward section of the strap's outer surface. This locks the brace securely in place both above and below the knee of the wearer. See FIGS. 5, 6 and 7.

It should be understood that various other types of two-part connectors may be employed for both fastening the first flap and releasably interengaging the upper and lower locking straps to themselves. Such two-part connector systems may include snap fasteners, buttons and complementary buttonholes, etc. Hook and loop connective systems are especially preferred.

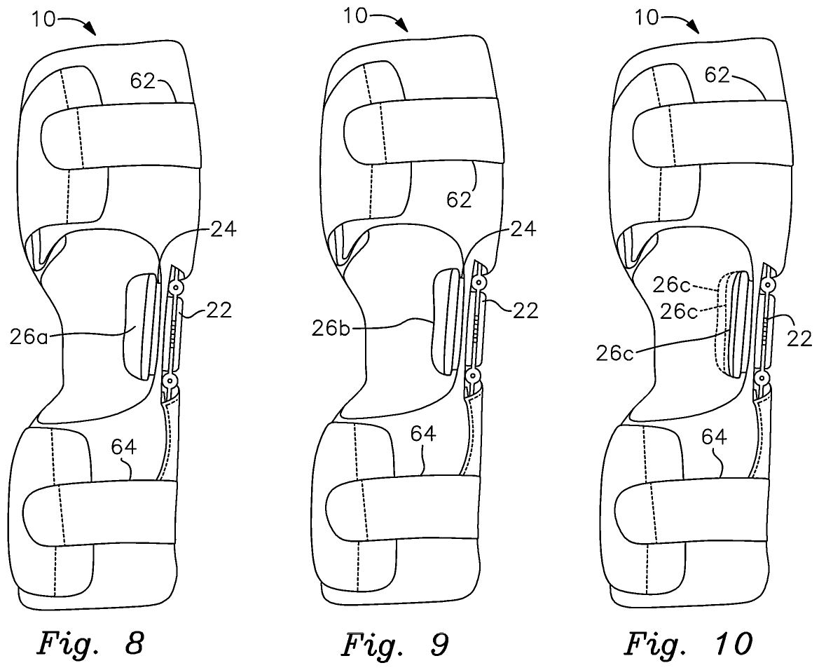

FIGS. 8, 9 and 10 disclose the knee brace 10 in a closed condition. As shown therein, a condyle pad 26a, 26b and 26c is secured to an interior surface of the central body section 12 of brace 10. More particularly, each condyle pad is mounted by a condyle bracket 24 that is secured to the brace interiorly of the hinge 22. In accordance with this invention, various pad thicknesses may be employed. In particular, the selected pad may have a custom thickness to accommodate the respective knee of a particular user. Adjustable condyle thicknesses may also be employed in alternative embodiments. For example, as shown in FIG. 10, one or more condyle pads 26c may be stacked together within a pouch or enclosure attached to the condyle bracket. In some cases, Velcro™ interconnections may be made to stack and attach multiple pads together and thereby adjust the thickness accordingly. Mounting the condyle pad(s) interiorly of the brace achieves more effective pressure and pain relief to the wearer's knee.

The present invention employs a unique innovative and advantageous means of bi-directionally tightening and securing the knee brace about the wearer's leg and knee. By employing a flap that is tightened in a first direction and a separate locking strap that is tightened in the reverse direction, a more comfortable and secure custom fit is achieved. The brace better resists undesirable movement and migration along the wearer's leg particularly during rehabilitation, exercise and other activities. Attaching the brace to the wearer's knee and removing the brace are accomplished quickly, conveniently and comfortably. The brace is easily adjusted to fit virtually any type and size of user. The bi-directional D-ring construction allows the size to be adjusted quickly, conveniently and reliably. The brace also employs an ergonomic construction and presents an aesthetically improved appearance.

Accordingly, the present invention relates to a universal wraparound knee brace that is beneficial for use by persons suffering from osteoarthritis, as well as patients recuperating from surgery and various other knee injuries. While this detailed description has set forth particularly preferred embodiments of the apparatus of this invention, numerous modifications and variations of the structure of this invention, all within the scope of the invention, will readily occur to those skilled in the art. Accordingly, it is understood that this description is illustrative only of the principles of the invention and is not limitative thereof.

Although specific features of the invention are shown in some of the drawings and not others, this is for convenience only, as each feature may be combined with any and all of the other features in accordance with this invention.

1. A therapeutic wraparound knee brace for being worn by a person having a knee injury or ailment, the knee brace comprising:
a flexible body for wrapping around the wearer's knee, said body having a central section for extending generally vertically across a side of the wearer's knee; said body further including a corresponding pair of first and second upper flaps attached to and extending outwardly from respective opposite vertical edges of said central section; said body further including a corresponding pair of first and second lower flaps attached to and extending outwardly from said respective opposite vertical edges of said central section below said first and second upper flaps;
a first concave recess formed between said first upper flap and said first lower flap, and a second concave recess formed between said second upper flap and said second lower flap;
each of said first upper and lower flaps including a respective outer surface that carries a respective first connector, which said first connector is releasably interengageable with a respective complementary second connector carried by at least one of said respective outer surface and an outer surface of said central section of said body; each of said second upper and lower flaps having an outer surface that supports a respective interior D-ring; and
an elongate upper locking strap attached proximate an inner end thereof to said outer surface of said second upper flap and an elongate lower locking strap attached proximate an inner end thereof to said outer surface of said second lower flap such that said upper and lower locking straps are extendable outwardly from said second upper and lower flaps respectively;
said upper and lower locking straps having respective exterior D-rings attached thereto proximate said inner ends of said upper and lower locking straps; each of said upper and lower locking straps having an outer surface that carries a respective complementary pair of releasably interengageable third and fourth connectors.

2. The device of claim 1 in which said body includes an internal frame covered by a padded exterior cover.

3. The device of claim 1 in which said body includes a hinge that permits said body to be angularly adjusted along a vertical axis.

4. The device of claim 2 in which said body includes a hinge connected to said internal frame.

5. The device of claim 1 in which at least one of said first, second, third and fourth connectors includes hook and loop material.

6. The device of claim 1 further including a condyle pad mounted to said body and further including means for adjusting a thickness of said pad.

7. The device of claim 1 in which each said exterior D-ring is generally parallel to said interior D-ring secured by said respective flap supporting said exterior D-ring.

8. A therapeutic wraparound knee brace for being worn by a person having a knee injury or ailment, the knee brace comprising:
a flexible body for wrapping around the wearer's knee, said body having a central section for extending generally vertically across a side of the wearer's knee; said body further including a corresponding pair of first and second upper flaps attached to and extending outwardly from respective opposite vertical edges of said central section; said body further including a corresponding pair of first and second lower flaps attached to and extending outwardly from said respective opposite vertical edges of said central section below said first and second upper flaps;
a first concave recess formed between said first upper flap and said first lower flap, and a second concave recess formed between said second upper flap and said second lower flap;
each of said first upper and lower flaps including a respective outer surface that carries a respective first connector, which said first connector is releasably interengageable with a respective complementary second connector carried by at least one of said respective outer surface and an outer surface of said central section of said body; each of said second upper and lower flaps having an outer surface that supports a respective interior D-ring; and
an elongate upper locking strap attached proximate an inner end thereof to said outer surface of said second upper flap and an elongate lower locking strap attached proximate an inner end thereof to said outer surface of said second lower flap such that said upper and lower locking straps are extendable outwardly from said second upper and lower flaps respectively;
said upper and lower locking straps having respective exterior D-rings attached thereto proximate said inner ends of said upper and lower locking straps; each of said upper and lower locking straps having an outer surface that carries a respective complementary pair of releasably interengageable third and fourth connectors;
said body being secured about the wearer's knee by wrapping said first upper and lower flaps respectively above and below the knee, operatively interengaging said first upper and lower flaps with said respective interior D-rings supported by said corresponding second upper and lower flaps, and interengaging said complementary first and second releasable connectors; the brace being further secured about the wearer's knee by wrapping said upper and lower locking straps respectively above and below the wearer's knee, operatively engaging each said locking strap with a respective said exterior D-ring and releasably interengaging said complementary third and fourth releasable connectors.

9. The device of claim 8 in which said body includes an internal frame covered by a padded exterior cover.

10. The device of claim 8 in which said body includes a hinge that permits the body to be angularly adjusted along a vertical axis.

11. The device of claim 9 in which said body includes a hinge connected to said internal frame.

12. The device of claim 8 in which at least one of said first, second, third and fourth connectors includes hook and loop material.

13. The device of claim 8 further including a condyle pad mounted to said body and further including means for adjusting a thickness of said pad.

14. The device of claim 8 in which each said exterior D-ring is generally parallel to said interior D-ring secured by said respective flap supporting said exterior D-ring.
